(19) United States
(12) Patent Application Publication
(10) Pub. No.: US 2025/0210135 A1
(43) Pub. Date: Jun. 26, 2025

(54) SYSTEMS AND METHODS FOR MONITORING EFFICACY OF CYTOTOXIC TREATMENT

(71) Applicant: Washington University, St. Louis, MO (US)

(72) Inventors: Aadel Chaudhuri, St. Louis, MO (US); Jose Zevallos, St. Louis, MO (US)

(21) Appl. No.: 18/848,363

(22) PCT Filed: Mar. 24, 2023
(86) PCT No.: PCT/US2023/016279

§ 371 Date: Sep. 18, 2024

Related U.S. Application Data
(60) Provisional application No. 63/323,920, filed on Mar. 25, 2022.

(51) Int. Cl. G16B 30/00; G16H 10/60; G16H 20/10; G16H 50/30

(52) U.S. Cl. CPC G16B 30/00; G16H 10/60; G16H 20/10; G16H 50/30

(57) ABSTRACT

A method of detecting the efficacy of a cytotoxic treatment for cancer in a patient is disclosed that includes comparing first and second tumor signals detected from first and second biofluids samples using ultra-low-pass whole genome sequencing (ULP-WGS) to obtain a tumor signal increase, and determining an efficacy of the cytotoxic treatment based on the tumor signal increase. The first and second biofluid samples are obtained from the patient prior to and after initiation of the cytotoxic treatment, respectively. A tumor signal increase exceeding a threshold level is indicative of the efficacy of the cytotoxic treatment.

### CROSS-REFERENCE TO RELATED APPLICATIONS

This application claims the benefit of U.S. Provisional Patent Application Ser. No. 63/323,920, filed Mar. 25, 1922, the disclosure of which is hereby incorporated by reference in its entirety.

### STATEMENT REGARDING FEDERALLY SPONSORED RESEARCH OR DEVELOPMENT

This work was supported by the U.S. Department of Veterans Affairs, and the Federal Government has certain rights in this invention.

### FIELD OF THE INVENTION

The present disclosure generally relates to systems and methods for monitoring the efficacy of a cytotoxic treatment in a subject.

### BACKGROUND

Timely and sensitive tracking of the progression of cancer and/or the response of cancer to treatment is an ongoing challenge in oncology. In some cases, patients are monitored by serial clinical and imaging exams, but this approach may lack sufficient sensitivity to capture changes that unambiguously indicate the progression of cancer or the response of cancer to a course of treatment, such as a cytotoxic treatment. Biopsies are also performed to diagnose and monitor cancer, but biopsies may similarly lack sensitivity and further are vulnerable to uncertainty due to geographic heterogeneity (biopsy of benign vs. malignant region of a tumor). In addition, biopsies can be morbid, painful, and not practical to perform for serial surveillance.

### SUMMARY OF THE INVENTION

In some aspects, a method of detecting an efficacy of a cytotoxic treatment for a cancer in a patient is disclosed that includes comparing first and second tumor signals detected from first and second biofluids samples, respectively to obtain a tumor signal increase, wherein the first and second biofluid samples are obtained from the patient prior to and after initiation of the cytotoxic treatment, respectively. The disclosed method further includes determining an efficacy of the cytotoxic treatment based on the tumor signal increase, wherein the tumor signal increase exceeding a threshold level is indicative of the efficacy of the cytotoxic treatment. In some aspects, the first and second tumor signals each comprise a tumor fraction, copy number alterations, genomic rearrangements, nucleotide variations, insertion/deletions, and any combination thereof. In some aspects, the method also includes obtaining or providing the first and second biofluid samples from the patient. In some aspects, the first and second biofluid samples comprise any one of blood samples, surgical drain fluid samples, urine samples, peritoneal fluid samples, saliva samples, and CSF samples. In some aspects, the second biofluid sample is obtained from about 2 hours to about 48 hours after initiation of the cytotoxic treatment. In some aspects, the method also includes isolating first and second amounts of cell-free DNA (cfDNA) from the first and second biofluid samples, respectively; performing ultra-low-pass whole genome sequencing (ULP-WGS) on the first and second amounts of cfDNA to obtain first and second pluralities of reads, respectively; and producing the first and second tumor signals based on the first and second pluralities of reads, respectively. In some aspects, performing ULP-WGS includes fragmenting the first and second amounts of cfDNA to obtain first and second pluralities of cfDNA fragments, respectively; constructing first and second DNA libraries comprising the first and second pluralities of cfDNA fragments, respectively; and sequencing the cfDNA fragments of the first and second DNA libraries to obtain first and second pluralities of reads, respectively, wherein each read comprises a read sequence and a read fragment size corresponding to each cFDNA fragment. In some aspects, the method also includes aligning each read sequence of the first and second pluralities of reads to a reference human genome to obtain first and second pluralities of aligned reads, respectively, and first and second tumor-specific genomic mutations, respectively; estimating first and second pluralities of local copy numbers based on the first and second pluralities of aligned reads, respectively; estimating first and second pluralities of copy number alterations by comparing the first and second pluralities of local copy numbers to a plurality of reference copy numbers, wherein the reference copy numbers comprise local copy numbers obtained from a population of control patients; and estimating first and second tumor signals based on the first and second pluralities of copy number alterations, respectively and first and second tumor-specific genomic mutations, respectively.

In another aspect, a method of monitoring an efficacy of a cytotoxic treatment for a cancer in a patient is disclosed that includes obtaining a baseline tumor signal and at least two post-treatment tumor signals detected from a baseline biofluid sample and at least two post-treatment biofluid samples, respectively, wherein the baseline biofluid sample is obtained from the patient prior to the cytotoxic treatment and the at least two post-treatment biofluid samples are obtained from the patient at different times after initiation of the cytotoxic treatment, respectively. The method further includes estimating a tumor signal kinetic characteristic based on the time sequence comprising the baseline tumor signal, the at least two post-treatment tumor signals, and associated times at which the baseline and at least two post-treatment biofluid samples were collected. The method further includes determining an efficacy of the cytotoxic treatment based on the tumor signal kinetic characteristic. In some aspects, the baseline and at least two post-treatment tumor signals each comprise a tumor fraction, copy number alterations, genomic rearrangements, nucleotide variations, insertion/deletions, and any combination thereof. In some aspects, the tumor signal kinetic characteristic comprises maximum tumor signal, rate of increase of tumor signal, time to maximum tumor signal, area under the curve from baseline to maximum tumor signal, and any combination thereof. In some aspects, the method further includes obtaining or providing the baseline and at least two post-treatment biofluid samples from the patient. In some aspects, the method further includes obtaining or providing the baseline and at least two post-treatment biofluid samples from the patient. In some aspects, the baseline and at least two post-treatment biofluid samples comprise any one of blood samples, surgical drain fluid samples, urine samples, peritoneal fluid samples, saliva samples, and CSF samples. In some aspects, the at least two post-treatment biofluid samples are obtained from about 2 hours to about 48 hours after initiation of the cytotoxic treatment. In some aspects, the method further includes isolating a baseline and at least two post-treatment amounts of cell-free DNA (cfDNA) from the baseline and at least two post-treatment biofluid samples, respectively; performing ultra-low-pass whole genome sequencing (ULP-WGS) on the baseline and at least two post-treatment amounts of cfDNA to obtain a baseline and at least two post-treatment pluralities of reads, respectively; and producing the baseline and at least two post-treatment tumor signals based on the baseline and at least two post-treatment pluralities of reads, respectively. In some aspects, performing ULP-WGS comprises: fragmenting the baseline and at least two post-treatment amounts of cfDNA to obtain baseline and at least two post-treatment pluralities of cfDNA fragments, respectively; constructing baseline and at least two post-treatment DNA libraries comprising the baseline and at least two post-treatment pluralities of cfDNA fragments, respectively; and sequencing the cfDNA fragments of the baseline and at least two post-treatment DNA libraries to obtain baseline and at least two post-treatment pluralities of reads, respectively, each read comprising a read sequence and a read fragment size corresponding to each cfDNA fragment. In some aspects, the method further includes aligning each read sequence of the baseline and at least two post-treatment pluralities of reads to a reference human genome to obtain baseline and at least two post-treatment pluralities of aligned reads, respectively and baseline and at least two post-treatment tumor-specific genomic mutations, respectively; estimating baseline and at least two post-treatment pluralities of local copy numbers based on the baseline and at least two post-treatment pluralities of aligned reads, respectively; estimating baseline and at least two post-treatment pluralities of copy number alterations by comparing the baseline and at least two post-treatment pluralities of local copy numbers to a plurality of reference copy numbers, wherein the reference copy numbers comprise local copy numbers obtained from a population of control patients; and estimating baseline and at least two post-treatment tumor signals based on the baseline and at least two post-treatment pluralities of copy number alterations, respectively and baseline and at least two post-treatment tumor-specific genomic mutations, respectively.

In an additional aspect, a method of predicting a responsiveness of cancer patient to a cytotoxic treatment is disclosed that includes obtaining a baseline tumor signal and at least one post-treatment tumor signal detected from a baseline biofluid sample and at least one post-treatment biofluid sample, respectively, wherein the baseline biofluid sample is obtained from the patient prior to the cytotoxic treatment and the at least one post-treatment biofluid sample is obtained from the patient after initiation of the cytotoxic treatment, respectively. The method further includes estimating a tumor signal kinetic characteristic based on the time sequence comprising the baseline tumor signal, the at least one post-treatment tumor signal, and associated times at which the baseline and at least one post-treatment biofluid samples were collected; and predicting the responsiveness of the patient to the cytotoxic treatment based on the tumor signal kinetic characteristic. In some aspects, the baseline and at least one post-treatment tumor signals each comprise a tumor fraction, copy number alterations, genomic rearrangements, nucleotide variations, insertion/deletions, and any combination thereof. In some aspects, the tumor signal kinetic characteristic comprises maximum tumor signal, rate of increase of tumor signal, time to maximum tumor signal, area under the curve from baseline to maximum tumor signal, and any combination thereof. In some aspects, the method further includes obtaining or providing the baseline and at least one post-treatment biofluid sample from the patient. In some aspects, the method further includes obtaining or providing the baseline and at least one post-treatment biofluid samples from the patient. In some aspects, the baseline and at least one post-treatment biofluid samples comprise any one of blood samples, surgical drain fluid samples, urine samples, peritoneal fluid samples, saliva samples, and CSF samples. In some aspects, the at least one post-treatment biofluid sample is obtained from about 2 hours to about 48 hours after initiation of the cytotoxic treatment. In some aspects, the method further includes isolating a baseline and at least one post-treatment amount of cell-free DNA (cfDNA) from the baseline and at least one post-treatment biofluid samples, respectively; performing ultra-low-pass whole genome sequencing (ULP-WGS) on the baseline and at least one post-treatment amounts of cfDNA to obtain a baseline and at least one post-treatment pluralities of reads, respectively; and producing the baseline and at least one post-treatment tumor signals based on the baseline and at least one post-treatment pluralities of reads, respectively. In some aspects, performing ULP-WGS includes fragmenting the baseline and at least one post-treatment amount of cfDNA to obtain baseline and at least one post-treatment pluralities of cfDNA fragments, respectively; constructing baseline and at least one post-treatment DNA libraries comprising the baseline and at least one post-treatment pluralities of cfDNA fragments, respectively; and sequencing the cfDNA fragments of the baseline and at least one post-treatment DNA libraries to obtain baseline and at least one post-treatment pluralities of reads, respectively, each read comprising a read sequence and a read fragment size corresponding to each cfDNA fragment. In some aspects, the method further includes aligning each read sequence of the baseline and at least one post-treatment pluralities of reads to a reference human genome to obtain baseline and at least one post-treatment pluralities of aligned reads, respectively and baseline and at least one post-treatment tumor-specific genomic mutations, respectively; estimating baseline and at least one post-treatment pluralities of local copy numbers based on the baseline and at least one post-treatment pluralities of aligned reads, respectively; estimating baseline and at least one post-treatment pluralities of copy number alterations by comparing the baseline and at least one post-treatment pluralities of local copy numbers to a plurality of reference copy numbers, wherein the reference copy numbers comprise local copy numbers obtained from a population of control patients; and estimating baseline and at least one post-treatment tumor signals based on the baseline and at least one post-treatment pluralities of copy number alterations, respectively and baseline and at least two post-treatment tumor-specific genomic mutations, respectively.

Other objects and features will be in part apparent and in part pointed out hereinafter.

### DESCRIPTION OF THE DRAWINGS

FIG. 1 is a graph summarizing the changes in tumor signals (HPV copies) over time for patients receiving a cytotoxic treatment at t=0.

Those of skill in the art will understand that the drawings, described below, are for illustrative purposes only. The drawings are not intended to limit the scope of the present teachings in any way.

### DETAILED DESCRIPTION OF THE INVENTION

In various aspects, a method of monitoring the efficacy of a cancer treatment including, but not limited to, a cytotoxic treatment is disclosed. The disclosed method enables early and sensitive detection of a response of a subject diagnosed with a cancer disorder to treatment with a cytotoxic treatment. The disclosed method makes use of a bio-fluids-based liquid biopsy approach to detect changes in a tumor signal obtained by the analysis of serial biofluid samples obtained from a subject before and after initiation of a cytotoxic treatment. The method makes use of the discovery that abrupt and short-term increases in the tumor signal relative to untreated baseline levels are observed in the biofluids of subjects after initiation of a cytotoxic treatment.

In various aspects, the liquid biopsy method includes the preparation of DNA isolated from a biofluid sample, followed by next-generation sequencing and a customized bioinformatics analysis approach to determine a tumor signal within the biofluid sample. Any suitable tumor signal may be obtained and monitored for the method as disclosed herein including, but not limited to, tumor fraction, tumor level, tumor-related copy number alterations and structural variations, and any other quantity indicative of an abundance of cell-free tumor DNA.

The disclosed method detects tumor genomic and epigenomic events in plasma. Traditional invasive tumor-based methods have shortcomings, including geographic tumor heterogeneity, such that important tumor clones can be missed in the biopsy specimen, leading to the incorrect conclusion upon analysis. The disclosed approach is based on analyzing tumor genomic and epigenomic events through biofluid (i.e. blood, urine) analysis, where geographic tumor heterogeneity should not be an issue. The disclosed novel approach can be used to flexibly detect tumor genomic and epigenomic alterations from nearly any bio-fluid or tissue type.

The disclosed method enables simultaneous assessment of genomic and epigenomic alterations including copy number alterations, genomic rearrangements, nucleotide variations, and insertions/deletions. In this way, we can query the oncogenomic status of a patient's tumor through the disclosed liquid biopsy approach. The disclosed method can be used to identify cancer early, as well as track response to treatment and identify recurrence early. We do this by applying the disclosed assay at serial timepoints to monitor genomic/epigenomic changes that occur over time.

Cell-free DNA (or cfDNA) refers to all non-encapsulated DNA in the bloodstream or other biofluid. A portion of that cell-free DNA originates from a tumor clone and is called circulating tumor DNA (or ctDNA). cFDNA includes nucleic acid fragments that enter the bloodstream during apoptosis or necrosis. Normally, these fragments are cleaned up by macrophages, but it is believed that the overproduction of cells in cancer leaves more of the cfDNA behind. These fragments average around 170 bases in length, have a half-life of about two hours in the bloodstream, and are present in both early and late-stage disease in many common tumors including non-small cell lung and breast. That said, cfDNA concentration varies greatly, for example occurring at between 1 and 100,000 fragments per milliliters of plasma.

Circulating tumor DNA (ctDNA) is found in the bloodstream and refers to DNA that comes from cancerous cells and tumors. Most DNA is inside a cell's nucleus. As a tumor grows, cells die and are replaced by new ones. The dead cells get broken down and their contents, including DNA, are released into the bloodstream. ctDNA includes small pieces of DNA, usually comprising fewer than 200 building blocks (nucleotides) in length.

In various aspects, whole-genome sequencing may include targeted sequencing using any known method without limitation including, but not limited to, a targeted sequencing method to sample limited regions of the genome known to contain alterations due to cancer. Without being limited to any particular theory, the targeted sequencing enhances the sensitivity of the results by selectively sequencing those portions of the genome associated with the transformation from a non-cancerous condition to a cancer condition.

In various aspects, the reads resulting from WGS are further analyzed to assess copy number variations relative to copy numbers associated with a reference condition including, but not limited to, a healthy normal condition or a cancer condition.

In various additional aspects, the copy number variations may be used to estimate a tumor fraction using any known method without limitation including, but not limited to, the use of software such as ichorCNA. In various aspects, the estimated tumor fraction is compared to a threshold value to determine whether the patient is at risk for the development of cancer or the extent of cancer in the patient. In some aspects, serial samples may be obtained and analyzed, and the resulting estimated tumor fractions may be compared to determine if the tumor fraction increased or decreased over time, indicating progression or remission of cancer, respectively. In other aspects, serial blood samples may be obtained and analyzed during various time points over a course of treatment to assess the efficacy of a treatment, and treatments may be terminated, altered, or continued based on changes in successive estimates of tumor fractions. In other additional aspects, serial samples may be obtained and analyzed to monitor for reoccurrence of cancer in a patient that is currently in remission.

The disclosed method enables simultaneous tracking of genomic events relevant to the patient's solid tumor malignancy (mutations, fusions, copy number alterations) as well as clonal hematopoiesis (mutations in the genes DMT3A, TET2 and ASXL1). In this way, genomic events related to the patient's primary malignancy vs. potentially confounding clonal hematopoiesis mutations can be delineated.

Non-limiting examples of suitable liquid biopsy methods are described in PCT Application No. PCT/US2020/064321 (published as PCT International Publication No. WO/2021/119318), PCT Application No. PCT/US2022/012748, and U.S. patent application Ser. No. 17/586,676 the contents of which are incorporated herein by reference in their entirety.

By way of non-limiting example, low-pass whole-genome sequencing may be performed using a customized protocol for isolating, preparing, and sequencing cell-free DNA from biofluid samples obtained from cancer patients. An analysis is performed on the low-pass whole-genome sequencing to detect and quantify copy number alterations, insertions/deletions, nucleotide variants, and genomic rearrangements. This whole-genome assay relies primarily on genome-wide copy number analysis, and patients harboring cancer have a distinct aberrant profile compared to those harboring benign disease after applying the disclosed custom protocol. Tumor signals indicative of the state, stage, and/or extent of the cancer of the patient may be calculated based on at least a portion of the features of this aberrant profile. In one aspect, the aberrant profile includes, but is not limited to, genome-wide copy number aberrations.

In various aspects, the liquid biopsy method may be implemented to produce results with a quick turnaround time. Consequently, changes in tumor signals may be monitored essentially in real-time. In addition, because the biofluid samples analyzed to determine tumor signals are obtained non-invasively, serial samples may be obtained and analyzed to provide for nearly continuous monitoring of the tumor signal.

Without being limited to any particular theory, the cytotoxic treatment induces cell death and increases short-term circulating tumor DNA (ctDNA) signals in blood plasma and other biofluids. The methods described herein make use of the discovery that cytotoxic treatment is accompanied by a short-term and sharp increase in the tumor signal (i.e. ctDNA) in surgical drain fluid and other biofluids as described herein. Non-limiting examples of suitable biofluid samples for monitoring the efficacy of cytotoxic treatments using the disclosed method include surgical drain fluid, urine, peritoneal fluid, saliva, CSF, and any other suitable biofluid.

The efficacy of any type of cytotoxic therapy may be monitored using the methods disclosed herein. Non-limiting examples of cytotoxic therapies that may be monitored using the disclosed methods include chemotherapy, radiotherapy, immunotherapy, and any combination thereof.

In various aspects, the methods disclosed herein compare tumor signals obtained prior to treatment (baseline) and at least one time after initiation of a cytotoxic treatment. In various aspects, the time series of pre-treatment and post-treatment tumor signals are further analyzed to determine a tumor signal kinetic characteristic that is indicative of the efficacy of the treatment. Any suitable tumor signal kinetic characteristic may be calculated and used to estimate the treatment efficacy. Non-limiting examples of suitable tumor signal kinetic characteristics include maximum tumor signal, rate of increase of tumor signal, time to maximum tumor signal, area under the curve from baseline to maximum tumor signal, and any combination thereof. By way of non-limiting example, a single baseline tumor signal and a post-treatment tumor signal obtained at a predetermined time after initiation of treatment are used to determine a rate of increase of tumor signal.

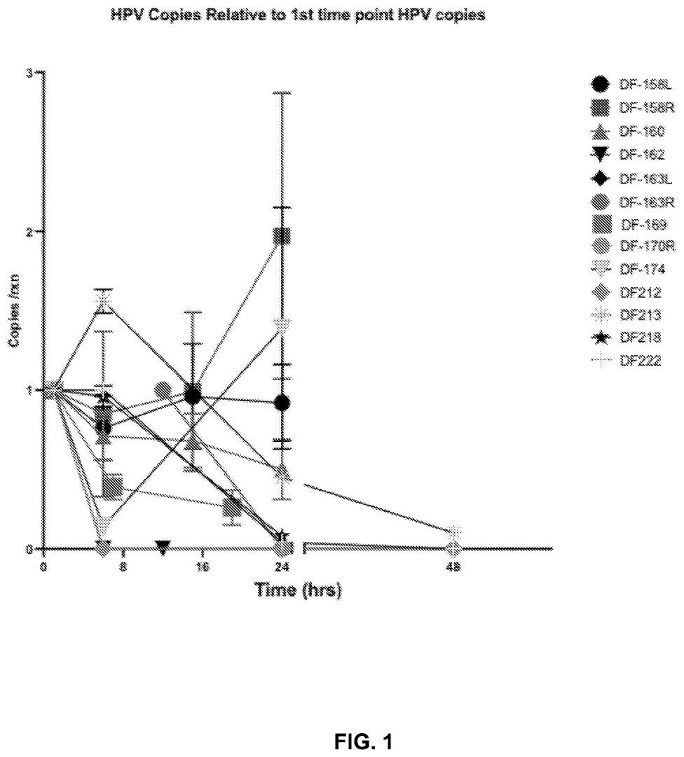

FIG. 1 Is a graph summarizing the tumor signals measured for a population of patients pre-treatment (t=0) and at several post-treatment times. Referring to FIG. 1, patient DF213 (grey asterisks) produced an initial increase in tumor signal six hours after initiation of treatment, which indicated the efficacy of the treatment. In various aspects, the time histories of tumor signals obtained from a population of patients receiving cytotoxic treatments may be analyzed along with clinically-obtained outcomes to define kinetic parameters and associated thresholds used to estimate the efficacy of the cytotoxic treatment. In various other aspects, the time histories of tumor signals obtained from a population of patients receiving cytotoxic treatments may be analyzed along with clinically-obtained outcomes to identify tumor signal kinetic characteristics observable during early treatment that are predictive of a patient's responsiveness to the treatment and/or long-term outcome. By way of non-limiting example, a large immediate spike in the tumor signal may correlate with a better overall cell-kill/treatment response.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

Constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | |
| (Basic): | K R H |
| Negatively Charged | |
| (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| | Ile, Val, Met, Ala, |
| Leu (L) | Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor& Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoom and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application-specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for the storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independently and separate from other components and processes described herein. Each component and process can also be used in combination with other assembly packages and processes. The present aspects may enhance the functionality and functioning of computers and/or computer systems.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

Non-limiting examples are provided herein to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

1. A method of detecting an efficacy of a cytotoxic treatment for a cancer in a patient, the method comprising:
a. comparing first and second tumor signals detected from first and second biofluids samples, respectively to obtain a tumor signal increase, wherein the first and second biofluid samples are obtained from the patient prior to and after initiation of the cytotoxic treatment, respectively; and
b. determining an efficacy of the cytotoxic treatment based on the tumor signal increase, wherein the tumor signal increase exceeding a threshold level is indicative of the efficacy of the cytotoxic treatment.

2. The method of claim 1, wherein the first and second tumor signals each comprise a tumor fraction, copy number alterations, genomic rearrangements, nucleotide variations, insertion/deletions, and any combination thereof.

3. The method of claim 1, further comprising obtaining or providing the first and second biofluid samples from the patient.

4. The method of claim 3, wherein the first and second biofluid samples comprise any one of blood samples, surgical drain fluid samples, urine samples, peritoneal fluid samples, saliva samples, and CSF samples.

5. The method of claim 1, wherein the second biofluid sample is obtained from about 2 hours to about 48 hours after initiation of the cytotoxic treatment.

6. The method of claim 1, further comprising:
a. isolating first and second amounts of cell-free DNA (cfDNA) from the first and second biofluid samples, respectively;
b. performing ultra-low-pass whole genome sequencing (ULP-WGS) on the first and second amounts of cfDNA to obtain first and second pluralities of reads, respectively; and
c. producing the first and second tumor signals based on the first and second pluralities of reads, respectively.

7. The method of claim 6, wherein performing ULP-WGS comprises:
a. fragmenting the first and second amounts 5 of cfDNA to obtain first and second pluralities of cfDNA fragments, respectively;
b. constructing first and second DNA libraries comprising the first and second pluralities of cfDNA fragments, respectively; and
c. sequencing the cfDNA fragments of the first and second DNA libraries to obtain first and second pluralities of reads, respectively, each read comprising a read sequence and a read fragment size corresponding to each cfDNA fragment.

8. The method of claim 7, further comprising:
a. aligning each read sequence of the first and second pluralities of reads to a reference human genome to obtain first and second pluralities of aligned reads, respectively and first and second tumor-specific genomic mutations, respectively;
b. estimating first and second pluralities of local copy numbers based on the first and second pluralities of aligned reads, respectively;
c. estimating first and second pluralities of copy number alterations by comparing the first and second pluralities of local copy numbers to a plurality of reference copy numbers, wherein the reference copy numbers comprise local copy numbers obtained from a population of control patients; and
d. estimating first and second tumor signals based on the first and second pluralities of copy number alterations, respectively, and first and second tumor-specific genomic mutations, respectively.

9. A method of monitoring an efficacy of a cytotoxic treatment for a cancer in a patient, the method comprising:
a. obtaining a baseline tumor signal and at least two post-treatment tumor signals detected from a baseline biofluid sample and at least two post-treatment biofluid samples, respectively, wherein the baseline biofluid sample is obtained from the patient prior to the cytotoxic treatment and the at least two post-treatment biofluid samples are obtained from the patient at different times after initiation of the cytotoxic treatment, respectively; and
b. estimating a tumor signal kinetic characteristic based on the time sequence comprising the baseline tumor signal, the at least two post-treatment tumor signals, and associated times at which the baseline and at least two post-treatment biofluid samples were collected; and
c. determining an efficacy of the cytotoxic treatment based on the tumor signal kinetic characteristic.

10. The method of claim 9, wherein the baseline and at least two post-treatment tumor signals each comprise a tumor fraction, copy number alterations, genomic rearrangements, nucleotide variations, insertion/deletions, and any combination thereof.

11. The method of claim 9, wherein the tumor signal kinetic characteristic comprises maximum tumor signal, rate of increase of tumor signal, time to maximum tumor signal, area under the curve from baseline to maximum tumor signal, and any combination thereof.

12. The method of claim 9, further comprising obtaining or providing the baseline and at least two post-treatment biofluid samples from the patient.

13. (canceled)

14. The method of claim 12, wherein the baseline and at least two post-treatment biofluid samples comprise any one of blood samples, surgical drain fluid samples, urine samples, peritoneal fluid samples, saliva samples, and CSF samples.

15. The method of claim 12, wherein the at least two post-treatment biofluid samples are obtained from about 2 hours to about 48 hours after initiation of the cytotoxic treatment.

16. The method of claim 9, further comprising:
a. isolating a baseline and at least two post-treatment amounts of cell-free DNA (cfDNA) from the baseline and at least two post-treatment biofluid samples, respectively;
b. performing ultra-low-pass whole genome sequencing (ULP-WGS) on the baseline and at least two post-treatment amounts of cfDNA to obtain a baseline and at least two post-treatment pluralities of reads, respectively; and
c. producing the baseline and at least two post-treatment tumor signals based on the baseline and at least two post-treatment pluralities of reads, respectively.

17. The method of claim 16, wherein performing ULP-WGS comprises:
a. fragmenting the baseline and at least two post-treatment amounts of cfDNA to obtain baseline and at least two post-treatment pluralities of cfDNA fragments, respectively;
b. constructing baseline and at least two post-treatment DNA libraries comprising the baseline and at least two post-treatment pluralities of cfDNA fragments, respectively; and
c. sequencing the cfDNA fragments of the baseline and at least two post-treatment DNA libraries to obtain baseline and at least two post-treatment pluralities of reads, respectively, each read comprising a read sequence and a read fragment size corresponding to each cfDNA fragment.

18. The method of claim 17, further comprising:
a. aligning each read sequence of the baseline and at least two post-treatment pluralities of reads to a reference human genome to obtain baseline and at least two post-treatment pluralities of aligned reads, respectively and baseline and at least two posttreatment tumor-specific genomic mutations, respectively;
b. estimating baseline and at least two post-treatment pluralities of local copy numbers based on the baseline and at least two post-treatment pluralities of aligned reads, respectively;
c. estimating baseline and at least two post-treatment pluralities of copy number alterations by comparing the baseline and at least two post-treatment pluralities of local copy numbers to a plurality of reference copy numbers, wherein the reference copy numbers comprise local copy numbers obtained from a population of control patients; and
d. estimating baseline and at least two post-treatment tumor signals based on the baseline and at least two post-treatment pluralities of copy number alterations, respectively, and baseline and at least two post-treatment tumor-specific genomic mutations, respectively.

19. A method of predicting a responsiveness of cancer patient to a cytotoxic treatment, the method comprising:
a. obtaining a baseline tumor signal and at least one post-treatment tumor signal detected from a baseline biofluid sample and at least one post-treatment biofluid sample, respectively, wherein the baseline biofluid sample is obtained from the patient prior to the cytotoxic treatment and the at least one post-treatment biofluid sample is obtained from the patient after initiation of the cytotoxic treatment, respectively; and
b. estimating a tumor signal kinetic characteristic based on the time sequence comprising the baseline tumor signal, the at least one post-treatment tumor signal, and associated times at which the baseline and at least one post-treatment biofluid samples were collected; and
c. predicting the responsiveness of the patient to the cytotoxic treatment based on the tumor signal kinetic characteristic.

20. The method of claim 19, wherein the baseline and at least one post-treatment tumor signals each comprise a tumor fraction, copy number alterations, genomic rearrangements, nucleotide variations, insertion/deletions, and any combination thereof.

21. The method of claim 19, wherein the tumor signal kinetic characteristic comprises maximum tumor signal, rate of increase of tumor signal, time to maximum tumor signal, area under the curve from baseline to maximum tumor signal, and any combination thereof.

22.-28. (canceled)
